(19) United States
(12) Patent Application Publication
(10) Pub. No.: US 2024/0081374 A1
(43) Pub. Date: Mar. 14, 2024

(54) COMPOSITIONS AND METHODS UTILIZING SOUTHERN OCEAN MACKEREL OIL FOR PROVIDING HEALTH BENEFITS IN AN ANIMAL

(71) Applicant: Société des Produits Nestlé S.A., Vevey (CH)

(72) Inventor: John Kenyon Russell-Maynard, New South Wales (AU)

(21) Appl. No.: 18/449,980

(22) Filed: Aug. 15, 2023

Related U.S. Application Data
(60) Provisional application No. 63/406,583, filed on Sep. 14, 2022.

(51) Int. Cl. A23K 10/22; A23K 20/147; A23K 20/158; A23K 20/163; A23K 50/40; A61K 35/60

(52) U.S. Cl. CPC A23K 10/22; A23K 20/147; A23K 20/158; A23K 20/163; A23K 50/40; A61K 35/60

(57) ABSTRACT

The present disclosure provides compositions and methods for providing a health benefit to an animal utilizing southern ocean mackerel oil. For example, a pet food composition for treating an inflammatory condition in a companion animal can comprise southern ocean mackerel oil and a preservative.

### CROSS REFERENCE TO RELATED APPLICATION

This application claims priority to U.S. Provisional Application Ser. No. 63/406,583 filed Sep. 14, 2022 the disclosure of which is incorporated in its entirety herein by this reference.

### BACKGROUND

Omega-3-fatty acids are known to be beneficial to mammals. Such benefits include those found in U.S. Pat. No. 5,776,913 disclosing specific diets comprising omega-3 fatty acids that can ameliorate metabolic disturbances in animals with cancer, U.S. Pat. No. 9,119,826 disclosing specific diets comprising omega-3 fatty acids for treating cardiovascular disorders, U.S. Pub. No. 2004/0235948 disclosing specific diets comprising omega-3 fatty acids for treatment of diabetes, and U.S. Pat. No. 5,434,183 disclosing specific diets comprising omega-3 fatty acids for treatment of inflammation, including arthritis. Omega-3-fatty acids are typically, exemplified by eicosapentaenoic acid (EPA) and docosahexaenoic (DHA), a-linolenic acid, and octadecatetraenoic acid. EPA and DHA are generally considered to be the most important and significant of the long chain omega-3 fatty acids. Generally, these long chain fatty acids are present in relatively low, almost minute, quantities or completely absent in typical pet food compositions. The fatty acid composition of foods consumed by companion pets, for example dogs and cats, directly influences the biological levels of fatty acid found in their blood.

Natural products which have relatively high levels of omega-3 unsaturated fatty acids such as EPA and DHA can be derived from marine oils such as salmon, anchovy, sardine and menhaden. Such natural oils can be concentrated to even higher percentages of omega-3-fatty acids. However, such products also contain high amounts of omega-6 fatty acids which increase the level of eicosanoides and leucotrienes, which can provide overactive inflammatory and immunological reactions, leading to deleterious effects. Also, further processing of the oils can be cost prohibitive.

In particular, the omega-6 fatty acid arachidonic acid at low levels is a key nutrient for the health of mammals. However, at higher levels, it is known to cause deleterious overactive inflammatory and immunological reactions. This deleterious response is known to be modulated by the omega-3 fatty acid equivalent of arachidonic acid or this omega-3 fatty acid's pre-cursors.

There is, therefore, a need for methods and compositions providing health benefits utilizing omega-3 fatty acids in an efficacious and cost-effective manner avoiding the issues above.

### SUMMARY

In one embodiment, a composition for providing a health benefit to an animal can comprise southern ocean mackerel oil and a preservative.

In another embodiment, a method of manufacturing a pet food composition can comprise admixing southern ocean mackerel oil and a preservative with at least one of protein, fat, and carbohydrate to form the pet food composition.

In yet another embodiment, a method of providing a health benefit to an animal can comprise administering to an animal a food composition comprising southern ocean mackerel oil and a preservative.

In various embodiments, such health benefits can include treating or preventing inflammatory conditions, such as inflammation, arthritis, irritated skin, swollen gums, allergy, asthma, autoimmune diseases, coeliac disease, glomerulonephritis, hepatitis, inflammatory bowel disease, reperfusion injury, transplant rejection, gout, myositis, scleroderma, systemic lupus erythematosus, vasculitis, psoriasis; or treating or preventing other health conditions such as cancer, heart disease, diabetes, renal disorders, and cognitive disorders.

Other and further objects, features, and advantages of the invention will be readily apparent to those skilled in the art.

### DETAILED DESCRIPTION

### Definitions

The term “animal” means any animal that has a need for the health benefits disclosed herein, including human, avian, bovine, canine, equine, feline, hircine, lupine, murine, ovine, or porcine animals. In one aspect, the animal can be a mammal. In another aspect, the animal can be a companion animal.

The term “companion animal” means domesticated animals such as cats, dogs, rabbits, guinea pigs, ferrets, hamsters, mice, gerbils, horses, cows, goats, sheep, donkeys, pigs, and the like. In one aspect, the companion animal can be a canine. In another aspect, the companion animal can be a canine and/or a feline.

The term “eicosatetraenoic acid” or “ETA” refers to all-cis-8,11,14,17-eicosatetraenoic acid, PubChem CID 11722594, CAS Registry number 2091-26-1.

The term “southern ocean mackerel oil” or “Southern Ocean mackerel oil” refers to an oil derived from at least one of the following: Trachurus declivis (Greenback horse mackerel) and/or Trachurus murphyi (Chilean jack mackerel) and/or Scomber australasicus (Blue mackerel) farmed in the southern hemisphere, i.e., waters south of the equator. In one embodiment, the mackerel can be farmed from the waters south of 20° S latitude. In one aspect, the mackerel can be farmed in waters south of 40° S latitude. In some embodiments, the oil may be derived at least in part from other mackerel species including Trachurus novaezelandiae (Yellowtail horse mackerel), Scomberomorus commerson (Narrow-barred Spanish mackerel), Scomberomorus queenshandicus (Queensland school mackerel) and other representatives of the Scomber Scomberomorus, Grammatorcynus, Acanthocybium and Trachurus genus or even members of the Carangidae or Gempylidae families. Generally, the southern ocean mackerel oil has at least 3% of omega-3 eicosatetraenoic acid (ETA). In one embodiment, the oil can have at least 30% omega-3 fatty acids and no more than 5% omega-6 fatty acids. In other aspects, the oil can have at least 1% of steridonic acid (SDA), at least 8% of eicosapentanaeoic acid (EPA), at least 15% of docosahexaenoic acid (DHA), and/or at least 2% of docosapentaenoic acid (all-cis-7,10,13,16,19-docosapentaenoic acid) (DPA). Additionally, such mackerel oils can include fractionations, extracts, derivatives, by-products and further concentrates of oils from the mackerels identified herein.

The term “therapeutically effective amount” means an amount of a compound disclosed herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The terms “treating”, “treat”, and “treatment” embrace both preventative, i.e., prophylactic, and palliative treatment.

The term “in conjunction” means that the food composition, components thereof, or other compositions disclosed herein are administered to an animal (1) together in a single food composition or (2) separately at the same or different frequency using the same or different administration routes at about the same time or periodically. “Periodically” means that the food composition, components thereof, or other compositions are administered on a schedule acceptable for specific compounds or compositions. “About the same time” generally means that the food composition, components thereof, or other compositions are administered at the same time or within about 72 hours of each other.

The term “food” or “food product” or “food composition” means a product or composition that is intended for ingestion by an animal, including a human unless the context dictates otherwise, and provides nutrition to the animal.

The term “carbohydrate” refers to carbohydrates that are digestible, e.g., sugars and starches, and does not include fiber, e.g., cellulose or fermentable fibers.

The term “regular basis” means at least monthly administration and, in one aspect, at least weekly administration. More frequent administration or consumption, such as twice or three times weekly, can be performed in certain embodiments. In one aspect, an administration regimen can comprise at least once daily consumption.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of the food compositions, or components thereof, physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

The term “about” means plus or minus 20% of a numeric value; in one aspect, plus or minus 10%; in another aspect, plus or minus 5%; and in one specific aspect, plus or minus 2%. For example, in one aspect where about is plus or minus 20% of a numeric value, the phrase “from about 10% to about 20%” could include a range from 8% to 24% or 12% to 16%, include any subranges therein.

As used herein, embodiments, aspects, and examples using “comprising” language or other open-ended language can be substituted with “consisting essentially of” and “consisting of” embodiments.

The term “complete and balanced” when referring to a food composition means a food composition that contains all known required nutrients in appropriate amounts and proportions based on recommendations of recognized authorities in the field of animal nutrition and are therefore capable of serving as a sole source of dietary intake to maintain life or promote production, without the addition of supplemental nutritional sources. Nutritionally balanced pet food and animal food compositions are widely known and widely used in the art, e.g., complete and balanced food compositions formulated according to standards established by the Association of American Feed Control Officials (AAFCO). In one embodiment, the term “complete and balanced” can be defined according the published AAFCO definition as of Jan. 1, 2022.

All percentages expressed herein are by weight of the composition on a dry matter basis unless specifically stated otherwise. The skilled artisan will appreciate that the term “dry matter basis” means that an ingredient's concentration or percentage in a composition is measured or determined after any free moisture in the composition has been removed.

As used herein, ranges are used herein in shorthand, so as to avoid having to list and describe each and every value within the range. Any appropriate value within the range can be selected, where appropriate, as the upper value, lower value, or the terminus of the range.

All dosages used herein refer to the amount of an ingredient or component or active per body weight of the animal per time period unless the context dictates otherwise. For example. in one embodiment, the mackerel oil dosage can be expressed as mg/kgBW/day or g/kgBW/day.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise, the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### DETAILED DESCRIPTION

The present methods and compositions are based upon the discovery that southern ocean mackerel oil provides a unique blend of omega-3 and omega-6 fatty acids that can be utilized in foods in a more cost-effective manner to provide various health benefits. Specifically, in one embodiment the southern ocean mackerel oil can be farmed from the waters around Southern Australia.

Specifically, in one embodiment, a pet food composition for treating an inflammatory condition in a companion animal can comprise southern ocean mackerel oil and a preservative.

Generally, the southern ocean mackerel oil can be present in the food composition in a therapeutically effective amount to provide a desired health benefit to the animal. In one embodiment, the southern ocean mackerel oil is present in the composition in an amount from about 0.1% to about 10%. In other aspects, the southern ocean mackerel oil can be present in the composition from 0.1%, 0.5%, 1%, 1.5%, 2%, 2.5%, 3%, 3.5%, 4%, 4.5%, 5%, 5.5%, 6%, 6.5%, or 7% to about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10%.

Generally, the southern ocean mackerel oil can comprise at least 3% of eicosatetraenoic acid (ETA). Additionally, in some embodiments the oil can have a high ratio of omega-3 fatty acids to omega-6 fatty acids, e.g., from about 4:1 to about 8:1. In one embodiment, the southern ocean mackerel oil can be farmed from the waters of Southern Australia (Southern Australia mackerel oil) and can comprise at least 30% omega-3 fatty acids and no more than 5% omega-6 fatty acids. In one aspect, the Southern Australian mackerel oil can comprise at least 3% of eicosatetraenoic acid (ETA), at least 1% of steridonic acid (SDA), at least 8% of eicosapentaenoic acid (EPA), at least 15% of docosahexaenoic acid (DHA), and at least 2% of docosapentaenoic acid (DPA).

Generally, the present food compositions and methods can provide a health benefit to the animal including such conditions related to inflammatory conditions. In one embodiment, such conditions can be selected from the group consisting of inflammation, arthritis, irritated skin, swollen gums, allergy, asthma, autoimmune diseases, coeliac disease, glomerulonephritis, hepatitis, inflammatory bowel disease, reperfusion injury, transplant rejection, gout, myositis, scleroderma, systemic lupus erythematosus, vasculitis, and psoriasis. Further, the composition can treat various related conditions including a member selected from the group consisting of: cancer, heart disease, diabetes, renal disorders, and cognitive disorders. Such cognitive disorders include brain aging, age-related or age-associated cognitive impairment, including short-term memory loss, diminished capacity to learn, diminished rate of learning, diminished attention, diminished motor performance, and/or dementia.

Generally, the compositions can be administered in a therapeutically effective amount to provide a desired benefit. In one embodiment, a daily dosage can be in a range of about 0.06 mg to about 50 mg of southern ocean mackerel oil (SOMO) per kg of body weight per day (SOMO/kgBW/day) can be efficacious in alleviating the presently described disorders and conditions and/or symptoms associated with such disorders/conditions in companion animals and likewise would also maintain health. More preferably, a dosage range of about 6 to about 40 mg of SOMO/kgBW/day, and most preferably, a dosage range of about 9 to about 30 mg of SOMO/kgBW/day can be efficacious. As such, in one aspect, a dog can receive from about 0.01 g/day to about 1.5 g/day of SOMO. In one aspect, the dosage level for a cat can be the amount required to reach a level of 0.03 g/kgBW/day of SOMO, although higher amounts may also be appropriate.

In one embodiment, the dosage of ETA can range from 1.8 μg to about 1.5 mg per kg of body weight per day (ETA/kgBW/day). In another embodiment, the dosage of SDA can range from 0.6 μg to about 0.5 mg per kg of body weight per day (SDA/kgBW/day). In still another embodiment, the dosage of EPA can range from 4.8 μg to about 4 mg per kg of body weight per day (EPA/kgBW/day). In another embodiment, the dosage of DHA can range from 9 μg to about 7.5 mg per kg of body weight per day (DHA/kgBW/day). In yet another embodiment, the dosage of DPA can range from 1.2 μg to about 1 mg per kg of body weight per day (DPA/kgBW/day). For example, in one aspect, a typical 25 Kg Dog might receive on average a dose of about 45 μg to 37.5 mg of ETA; 15 μg to 12.5 mg of SDA; 120 μg to 100 mg of EPA; 225 μg to 188 mg of DHA; and g to 25 mg of DPA per day.

Additionally, the present compositions can be administered in amounts to provide a therapeutically effective amount of steridonic acid, eicosapentaenoic acid, docosahexaenoic acid, and/or docosapentaenoic acid to treat or alleviate the presently disclosed conditions/disorders.

In one embodiment, the amount of SOMO per 1000 Kcal of a pet food product for treating and/or alleviating the presently described disorders and conditions and/or symptoms associated with such disorders/conditions in companion animals can be generally about 3.7 mg to about 3000 mg of SOMO/1000 Kcal of pet food. More preferably, an amount of 35 mg to 2000 mg of SOMO/1000 Kcal and most preferably, an amount of 180 mg to 1500 mg of SOMO/1000 Kcal of a pet food product can be efficacious.

In other embodiments, other dosages can be generally about 0.11 mg to about 90 mg of ETA/1000 Kcal; about 0.03 mg to about 30 mg of SDA/1000 Kcal; about 0.29 mg to about 240 mg of EPA/1000 Kcal; about 0.55 mg to about 450 mg of DHA/1000 Kcal and/or about 0.07 mg to about 60 mg of DPA/1000 Kcal. More preferably, a dosage of about 1.05 mg to about 60 mg of ETA/1000 Kcal; about 0.35 mg to about 20 mg of SDA/1000 Kcal; about 2.8 mg to about 160 of EPA/1000 Kcal; about 5.25 mg to about 300 mg of DHA/1000 Kcal; and/or about 0.7 mg to about 40 mg of DPA/1000 Kcal. Most preferably, a dosage of about 5.4 mg to about 45 mg of ETA/1000 Kcal; about 1.8 mg to about 15 mg of SDA/1000 Kcal; about 14.4 mg to about 120 mg of EPA/1000 Kcal; about 27 mg to about 225 mg of DHA/1000 Kcal; and/or about 3.6 mg to about 30 mg of DPA/1000 Kcal.

The present composition can be any composition suitable for an animal including food compositions, supplements, treats, pharmaceutical compositions, and the like. In one embodiment, the composition can be a food composition. In one aspect, the composition can be a main meal composition. In another aspect, the composition can be a supplement. In one embodiment, the animal can be a human or companion animal. In one aspect, the animal can be a companion animal. In another aspect, the animal can be a feline. In one aspect, the animal can be a canine. In another aspect, the animal can be a human. As such, the present methods and compositions can be directed and/or formulated for such animals, e.g., formulated for a canine or feline.

In one embodiment, the compositions can be food compositions. In one aspect, the composition can further comprise protein, fat, carbohydrates, and fiber. In another embodiment, the composition can further comprise an additive effective for treating the presently disclosed conditions. Such additives can include any pharmacological treatment or nutritional ingredients.

Generally, the present food compositions comprise a protein. The protein can be crude protein material and may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include beef, pork, lamb, equine, poultry, fish, and mixtures thereof. In one embodiment, the food compositions can comprise the protein in amounts from about 15%, 20%, 25%, 30%, 35%, 40%, 45%, 50%, 55%, or even 60% to about 25%, 30%, 35%, 40%, 45%, 50%, 55%, 60%, or even 65%, including various subranges within these amounts. In one aspect, the protein can be from about 20% to about 50% of the food composition.

Generally, any type of carbohydrate can be used in the food compositions. Examples of suitable carbohydrates include grains or cereals such as rice, corn, millet, sorghum, alfalfa, barley, soybeans, canola, oats, wheat, rye, triticale and mixtures thereof. The compositions may also optionally comprise other materials such as dried whey and other dairy by-products. In one embodiment, the carbohydrate comprises from about 5% to about 70% of the food composition. In another embodiment, the carbohydrate comprises from about 20% to about 50% of the food compositions. In other aspects, the carbohydrate can be present in amounts from about 5%, 10%, 15%, 20%, 25%, 30%, 35%, or even 40%, to about 20%, 25%, 30%, 35%, 40%, 45%, 50%, 55%, 60, 65%, or 70%.

Generally, the food compositions include fat. Such fat can include the southern ocean mackerel oil or be in addition to the mackerel oil. Examples of suitable fats include animal fats and vegetable fats. In one aspect, the fat source can be an animal fat source such as tallow or poultry fat. Vegetable oils such as corn oil, sunflower oil, safflower oil, grape seed oil, soybean oil, olive oil and other oils rich in monounsaturated and polyunsaturated fatty acids, may also be used. In one embodiment, the food compositions can comprise the fat in amounts from about 10%, 15%, 20%, 25%, 30%, or even 35% to about 15%, 20%, 25%, 30%, 35%, 40%, 45%, or even 50%, including various subranges within these amounts. In one aspect, the fat comprises from about 5% to about 40% of the food composition.

The administration can be performed on as-needed basis, an as-desired basis, a regular basis, or intermittent basis. In one aspect, the food composition can be administered to the animal on a regular basis. In one aspect, at least weekly administration can be performed. In another aspect, an administration regimen can comprise at least once daily consumption. Generally, the compositions can be administered to the animal on a basis to achieve at least one of the health benefits disclosed herein or treat or alleviate one of the disorders/conditions disclosed herein, including symptoms of such disorders/conditions. In another embodiment, the composition can be administered on a long-term basis, i.e., for at least 6 months. In other aspects, the long-term basis can be for at least 1 year, 2, years, 3 years, or the life of the animal.

According to the presently described methods, administration, including administration as part of a dietary regimen, can span a period ranging from parturition through the adult life of the animal. In various embodiments, the animal can be a human or companion animal such as a dog or cat. In certain embodiments, the animal can be a young or growing animal. In other embodiments, administration can begin, for example, on a regular or extended regular basis, when the animal has reached more than about 10%, 20%, 30%, 40%, or 50% of its projected or anticipated lifespan. In some embodiments, the animal can have attained 40, 45, or 50% of its anticipated lifespan. In yet other embodiments, the animal can be older having reached 60, 66, 70, 75, or 80% of its likely lifespan. A determination of lifespan may be based on actuarial tables, calculations, estimates, or the like, and may consider past, present, and future influences or factors that are known to positively or negatively affect lifespan. Consideration of species, gender, size, genetic factors, environmental factors and stressors, present and past health status, past and present nutritional status, stressors, and the like may also influence or be taken into consideration when determining lifespan.

Such administration can be performed for a time required to accomplish one or more objectives described herein, e.g., treating or alleviating the conditions/disorders described herein or symptoms relating thereto. Other administration amounts may be appropriate and can be determined based on the animal's initial weight as well as other variables such as species, gender, breed, age, desired health benefit, etc.

The moisture content for such food compositions generally varies depending on the nature of the food composition. The food compositions may be dry compositions (e.g., kibble), semi-moist compositions, wet compositions, or any mixture thereof. In one embodiment, the composition can be a pet food composition, and in one aspect, can be a complete and nutritionally balanced pet food. In this embodiment, the pet food may be a “wet food”, “dry food”, or food of “intermediate moisture” content. “Wet food” describes pet food that is typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one embodiment, the compositions can have moisture content from about 5% to about 20%. Dry food products include a variety of foods of various moisture contents, such that they are relatively shelf-stable and resistant to microbial or fungal deterioration or contamination. Also, in one aspect, dry food compositions can be extruded food products for either humans or companion animals.

The food compositions may also comprise one or more fiber sources. Such fiber sources include fiber that is soluble, insoluble, fermentable, and nonfermentable. Such fibers can be from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefits described herein or to the immune system of an animal.

In some embodiments, the ash content of the food composition ranges from less than 1% to about 15%. In one aspect, the ash content can be from about 5% to about 10%.

Generally, the food composition can be suitable for consumption by an animal, including humans and companion animals such as dogs and cats, as a meal, component of a meal, a snack, or a treat. Such compositions can include complete foods intended to supply the necessary dietary requirements for an animal. Examples of such food compositions include but are not limited to dry foods, wet foods, drinks, bars, frozen prepared foods, shelf prepared foods, and refrigerated prepared foods.

Food compositions may further comprise one or more substances such as vitamins, minerals, antioxidants, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat-soluble vitamins as A, D, E, and K. Inulin, amino acids, enzymes, coenzymes, and the like may be useful to include in various embodiments.

In various embodiments, the food compositions can contain enhanced levels of at least one or more antioxidants and in particular natural antioxidants such as tocopherols including vitamin E, flavonoids including anthocyanins; carotenoids including Astaxanthin, lutein and zeaxanthin; phenolics and polyphenolics, Rosemary and other plant extracts. The presence of enhanced levels of antioxidants helps protect the southern ocean mackerel oil from oxidation and helps ensure it is efficacious until the end of a product's shelf life.

In various embodiments, the food compositions contain at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the food compositions (e.g., uniformly or non-uniformly distributed in the compositions) or applied to the food compositions (e.g., topically applied with or without a carrier). Such methods are known to skilled artisans, e.g., U.S. Pat. No. 5,968,569 and related patents.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp.

Generally, the food compositions can contain probiotics in amounts sufficient to supply from about 10⁴ to about 10¹² cfu/animal/day, in one aspect, from 10⁵ to about 10¹¹ cfu/animal/day, and in one specific aspect, from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In another embodiment, the probiotics can be encapsulated in a carrier using methods and materials known to skilled artisans.

As stated, the food compositions may contain one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. In one embodiment, the prebiotic can be chicory root, chicory root extract, inulin, or combinations thereof. Generally, prebiotics can be administered in amounts sufficient to positively stimulate the healthy microflora in the gut and cause these “good” bacteria to reproduce. Typical amounts range from about one to about 10 grams per serving or from about 5% to about 40% of the recommended daily dietary fiber for an animal. The probiotics and prebiotics can be made part of the composition by any suitable means. Generally, the agents can be mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, in one aspect, from about 0.3 to about 7%, and in one specific aspect, from about 0.5 to 5%, on a dry matter basis. The prebiotics can be integrated into the compositions using methods known to skilled artisans, e.g., U.S. Pat. No. 5,952,033.

A skilled artisan can determine the appropriate amount of food ingredients, vitamins, minerals, probiotics, prebiotics, antioxidants, or other ingredients to be used to make a particular composition to be administered to a particular animal. Such artisan can consider the animal's species, age, size, weight, health, and the like in determining how best to formulate a particular composition comprising such ingredients. Other factors that may be considered include the desired dosage of each component, the average consumption of specific types of compositions by different animals (e.g., based on species, body weight, activity/energy demands, and the like), and the manufacturing requirements for the composition.

In a further aspect, the present disclosure provides kits suitable for administering food compositions to animals. The kits comprise in separate containers in a single package or in separate containers in a virtual package, as appropriate for the kit component, one or more of (1) one or more ingredients suitable for consumption by an animal; (2) instructions for how to combine the ingredients and other kit components to produce a composition useful for providing a health benefit as described herein; (3) instructions for how to use the food composition to obtain such benefits; (4) one or more probiotics; (5) one or more inactivated probiotics; (6) one or more components of inactivated probiotics that promote health benefits similar to or the same as the probiotics, e.g., proteins, lipids, glycoproteins, and the like; (7) one or more prebiotics; (8) a device for preparing or combining the kit components to produce a composition suitable for administration to an animal; and (9) a device for administering the combined or prepared kit components to an animal. In one embodiment, the kit comprises one or more ingredients suitable for consumption by an animal. In another embodiment, the kit comprises instructions for how to combine the ingredients to produce a composition useful for obtaining a health benefit as described herein.

When the kit comprises a virtual package, the kit is limited to instructions in a virtual environment in combination with one or more physical kit components. The kit contains components in amounts sufficient for to obtain a health benefit as described herein. Typically, the kit components can be admixed just prior to consumption by an animal. The kits may contain the kit components in any of various combinations and/or mixtures. In one embodiment, the kit contains a container of food for consumption by an animal. The kit may contain additional items such as a device for mixing ingredients or a device for containing the admixture, e.g., a food bowl. In another embodiment, the food compositions can be mixed with additional nutritional supplements such as vitamins and minerals that promote good health in an animal. The components can be each provided in separate containers in a single package or in mixtures of various components in different packages. In some embodiments, the kits comprise one or more other ingredients suitable for consumption by an animal. In one aspect, such kits can comprise instructions describing how to combine the ingredients to form a food composition for consumption by the animal, generally by mixing the ingredients or by applying optional additives to the other ingredients, e.g., by sprinkling nutritional supplements on a food composition.

In a further aspect, a means for communicating information about or instructions for one or more of (1) using a food composition for obtaining one of the health benefits described herein; (2) contact information for consumers to use if they have a question regarding the methods and compositions described herein; and (3) nutritional information about the food composition can be provided. The communication means can be useful for instructing on the benefits of using the present methods or compositions and communicating the approved methods for administering food compositions to an animal. The means comprises one or more of a physical or electronic document, digital storage media, optical storage media, audio presentation, audiovisual display, or visual display containing the information or instructions. In one aspect, the means can be selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

The present disclosure also contemplates methods of manufacturing food compositions. In one embodiment, a method of manufacturing a pet food composition can comprise admixing southern ocean mackerel oil and a preservative with at least one of protein, fat, and carbohydrate to form the food composition. In other aspects, methods for manufacturing a food composition can comprise one or more other ingredients suitable for consumption by an animal, e.g., protein, fat, carbohydrate, fiber, vitamins, minerals, probiotics, prebiotics, and the like, and can comprise admixing one or more of the ingredients suitable for consumption by an animal. The composition can be made according to any method suitable in the art, including admixing, pre-conditioning, extrusion, emulsification, etc.

Additionally, A method of treating an inflammatory condition comprising administering to an animal a food composition containing southern ocean mackerel oil, including any of those described herein.

In another aspect, a package useful for containing compositions described herein can comprise at least one material suitable for containing the food composition and a label affixed to the package containing a word or words, picture, design, acronym, slogan, phrase, or other device, or combination thereof that indicates that the contents of the package contains the food composition. In some embodiments, the label affixed to the package contains a word or words, picture, design, acronym, slogan, phrase, or other device, or combination thereof that indicates that the contents of the package contains the food composition with beneficial properties relating to a health benefit described herein. In one aspect, such device can comprise the words “for inflammation,” treats inflammation,” “delays onset of arthritis” or an equivalent or similar expression printed on the package. Any package configuration and packaging material suitable for containing the composition can be used herein, e.g., bag, box, bottle, can, pouch, and the like manufactured from paper, plastic, foil, metal, and the like. In one embodiment, the package contains a food composition adapted for a particular animal such as a human, canine, or feline, as appropriate for the label, in one aspect, a companion animal food composition for dogs or cats. In one embodiment, the package can be a can or pouch comprising a food composition described herein. In various embodiments, the package further comprises at least one window that permit the package contents to be viewed without opening the package. In some embodiments, the window can be a transparent portion of the packaging material. In others, the window can be a missing portion of the packaging material.

In an additional embodiment evident to those skilled in the art, the southern ocean mackerel oil can provide additional essential nutrients such as other essential fatty acids to help deliver complete and balanced nutrition as required by the cat or dog.

### EXAMPLES

The invention can be further illustrated by the following example, although it will be understood that this example is included merely for purposes of illustration and is not intended to limit the scope of the invention unless otherwise specifically indicated.

### Example 1—Pet Food with Southern Ocean Mackerel Oil

A pet food is manufactured by admixing the ingredients of Table 1 and then extruding to form a kibble. Optionally, the kibble can be further coated and/or dried to provide increased palatability and/or moisture properties.

TABLE 1

| Ingredients | Weight % |
|---|---|
| Protein | 20-65% |
| Carbohydrate | 10-50% |
| Fat | 5-30% |
| Fiber | 1-15% |
| Southern ocean mackerel oil | 0.1-10% |
| Vitamin/Mineral | 0.5-3% |
| Water | 0-10% |

The kibble can be administered to a companion animal to provide a therapeutically effective amount of southern ocean mackerel oil for providing various health benefits to the animal including inflammation, arthritis, irritated skin, swollen gums, allergy, asthma, autoimmune diseases, coeliac disease, glomerulonephritis, hepatitis, inflammatory bowel disease, reperfusion injury, transplant rejection, gout, myositis, scleroderma, systemic lupus erythematosus, vasculitis, and/or psoriasis.

In the specification, there have been disclosed certain embodiments of the invention. Although specific terms are employed, they are used in a generic and descriptive sense only and not for purposes of limitation. The scope of the invention is set forth in the claims. Obviously, many modifications and variations of the invention are possible in light of the above teachings. It is therefore to be understood that within the scope of the appended claims, the invention may be practiced otherwise than as specifically described.

What is claimed is:

1. A pet food composition for treating an inflammatory condition in a companion animal comprising southern ocean mackerel oil and a preservative.

2. The composition of claim 1, wherein the southern ocean mackerel oil is present in the composition in an amount from about 0.1% to about 10%.

3. The composition of claim 1, wherein the southern ocean mackerel oil comprises at least 30% omega-3 fatty acids and no more than 5% omega-6 fatty acids.

4. The composition of claim 1, wherein the southern ocean mackerel oil comprises at least 3% of eicosatetraenoic acid, at least 1% of steridonic acid, at least 8% of eicosapentanaeoic acid, at least 15% of docosahexaenoic acid, and at least 2% of docosapentaenoic acid.

5. The composition of claim 1, wherein the composition further comprises protein in an amount from about 20% to about 50%.

6. The composition of claim 1, wherein the composition further comprises carbohydrate in an amount of from about 5% to about 70%.

7. The composition of claim 1, wherein the composition further comprises fat in an amount of from about 5% to about 40%.

8. The composition of claim 1, wherein the composition is a main meal composition.

9. The composition of claim 1, wherein the composition is a supplement.

10. The composition of claim 1, wherein the inflammatory condition is selected from the group consisting of inflammation, arthritis, irritated skin, swollen gums, allergy, asthma, autoimmune diseases, coeliac disease, glomerulonephritis, hepatitis, inflammatory bowel disease, reperfusion injury, transplant rejection, gout, myositis, scleroderma, systemic lupus erythematosus, vasculitis, psoriasis.

11. The composition of claim 1, wherein the composition can treat a member selected from the group consisting of: cancer, heart disease, diabetes, renal disorders, and cognitive disorders.

12. A method of manufacturing a pet food composition comprising admixing southern ocean mackerel oil and a preservative with at least one of protein, fat, and carbohydrate to form the pet food composition.

13. The method of claim 12, wherein the southern ocean mackerel oil is present in the composition in an amount from about 0.1% to about 10%.

14. The method of claim 12, wherein the pet food composition is a main meal composition.

15. The method of claim 12, wherein the pet food composition is a supplement.
